(19) United States
(12) Patent Application Publication
(10) Pub. No.: US 2023/0302193 A1
(43) Pub. Date: Sep. 28, 2023

(54) PLASTER PREPARATION, POLYMER FILM, SPRAY PLASTER SPRAY DEVICE, METHOD FOR PRODUCING A POLYMER FIBER NONWOVEN MATERIAL, POLYMER FIBER NONWOVEN MATERIAL, AND USES OF THE PLASTER PREPARATION

(71) Applicant: PolyMedics Innovations GmbH, Denkendorf (DE)

(72) Inventors: Heinrich Planck, Nuertingen (DE); Erhard Mueller, Stuttgart (DE); Svenja Reimer, Aichtal (DE); Christian Planck, Kirchheim (DE); Lisa Lang, Stuttgart (DE)

(73) Assignee: PolyMedics Innovations GmbH, Denkendorf (DE)

(21) Appl. No.: 18/327,302

(22) Filed: Jun. 1, 2023

Related U.S. Application Data
(63) Continuation of application No. PCT/EP2021/084217, filed on Dec. 3, 2021.

(30) Foreign Application Priority Data
Dec. 3, 2020 (DE) 10 2020 215 294.2

(51) Int. Cl. A61L 26/00; A61F 15/00

(52) U.S. Cl. CPC A61L 26/0076; A61F 15/005; A61L 26/009; A61L 26/0019; A61L 26/0066; A61L 2300/404

(57) ABSTRACT

A plaster preparation includes (a) at least one resorbable polymer made of at least three monomer units, in particular a terpolymer, having monomers selected from the group consisting of trimethylene carbonate, glycolide, lactide (in particular DL-lactide), p-dioxanone, ε-caprolactone and/or butyrolactone; (b) at least one readily vaporizable organic solvent; and (c) at least one propellant. The plaster preparation additionally relates to a polymer film, to a spray plaster spray device, to a method for producing polymer fiber nonwoven materials, to a polymer fiber nonwoven material, and to uses of the plaster preparation.

### CROSS REFERENCE TO RELATED APPLICATIONS

This continuation application claims priority to PCT/EP2021/084217 filed on Dec. 3, 2021 which has published as WO 2022/117842 A1 and also the German application number 10 2020 215 294.2 filed on Dec. 3, 2020, the entire contents of which are fully incorporated herein with these references.

### DESCRIPTION

### Field of the Invention

The invention relates to a plaster preparation, a polymer film produced by means of spraying the plaster preparation, a spray plaster spray device for producing a polymer membrane or a polymer fiber nonwoven material, respectively, a method for producing the polymer fiber nonwoven material, a polymer fiber nonwoven material, and uses of the plaster preparation.

### Background of the Invention

Conventional spray plasters comprising acrylate-, silicone- or polyurethane-based polymers, a solvent, such as ethyl acetate, and a propellant, for example dimethyl ether, hydrocarbons, fluorine-containing hydrocarbons, CO2, nitrogen or noble gases, are known from the prior art. Disadvantages of such spray plasters include the physiological harmlessness of the polymers, their insufficient flexibility, particularly with thin films, and/or adhesion to skin or wounds, and insufficient water permeability, particularly due to high hydrophobicity.

EP 0 509 203 A2 is aimed at providing physiologically safe polymers that can be used on the skin without the disadvantages of known mono- and copolymers of lactic acid and glycolic acid. The known mono- and copolymers are usually applied in solutions and, in accordance with the disclosure of EP 0 509 203 A2, are inherently relatively hard and not very flexible, so that sprayed-on films quickly become brittle and cracked after evaporation of the solvent and have only limited adhesion to skin or wound surfaces.

Known polymers of lactide and ε-caprolactone are described in EP 0 509 203 A2 as generally rigid thermoplastics that are suitable for the production of containers, for example, but are unsuitable for topical application.

EP 0 509 203 A2 discloses for improvement the use of copolymers of racemic lactide and ε-caprolactone, δ-valerolactone, racemic γ-decalactone or β-hydroxybutyric acid, wherein the copolymers are prepared by conversion of the monomers in a lactide to reactant molar ratio of approximately 95 to 70 to 5 to 30 with the addition of metal carboxylates known per se as initiators at temperatures of approximately 150° C. for a period of approximately 16 to 48 hours. As an example, a solution of a D, L-lactide and ε-caprolactone copolymer in a molar ratio of 85 to 15 is disclosed, which can be filled into known pump sprays or aerosol sprays.

EP 1 077 073 B1 is directed at improving resorbable, physiologically safe copolylactides. EP 1 077 073 B1 discloses in this regard that application to uninjured skin requires copolylactides with high adherence, flexibility and extensibility. According to EP 1 077 073 B1, water vapor permeability is a crucial criterion for use on injured skin and, according to EP 1 077 073 B1, cannot be achieved by the copolymers alone because they are hydrophobic and have low permeability (<60 ml/h/m²). According to the disclosure of EP 1 077 073 B1, either a relatively high monomer content in the reaction product or the addition of hygroscopic/hydrophilic substances (e.g., glycerol) is required, through which a vapor permeability of initially high values (>150 ml/h/m²) is achieved, which decreases over the course of days (corresponding to wound secretion).

EP 1 077 073 B1 discloses copolymers of racemic lactide and the comonomers ε-caprolactone, δ-valerolactone, 1,4-dioxanone-2 or 1,3-dioxanone-2 in a lactide/comonomer molar ratio of 90-80/10-20, which were polymerized in the presence of tin(II) diethyl hexanoate as initiator and a cocatalyst at approximately 160° C., and have a glass transition temperature between 30 and 43° C., a molecular weight Mn of 15,000 to 50,000 and a polydispersity Pn (Mw/Mn) between 1.2 and 2. In particular, a copolymer of D,L-lactide and 1,3-dioxanone-2 (also known as trimethylene carbonate) prepared in this manner is disclosed, as is a copolymer of D,L-lactide and ε-caprolactone. In accordance with the teachings of EP 1 077 073 B1, a low viscosity leads to a sticky consistency, which must result in adhesion with overlying textiles (e.g., dressing material). Copolymers with the same lactide/comonomer ratio with high viscosity, on the other hand, are not very flexible, stiff, brittle and have poor adhesion, according to EP 1 077 073 B1. The target viscosity of the copolymers according to the invention in EP 1 077 073 B1 is between 0.30 and 0.75, preferably between 0.55 and 0.67.

### SUMMARY OF THE INVENTION

It is an object of the invention to provide a polymer-based plaster preparation suitable for producing both polymer films and polymer fiber nonwoven materials, wherein a high viscosity of the plaster preparation as well as a high extensibility of the polymer film or the polymer fiber nonwoven material, respectively, is achieved at low thickness, wherein both polymer films and polymer fiber nonwoven materials exhibit good adhesion to skin (human or animal, dry or wet) or wounds, and wherein a high water vapor permeability, in particular comparable to the water vapor permeability of skin (human or animal), is achieved without the need for further additives. Furthermore, a polymer film, a spray plaster spray device, and a method for producing a polymer nonwoven material are to be disclosed.

The object relating to the plaster preparation is solved according to the invention by a plaster preparation having the features of claim 1. The polymer film according to the invention has the features disclosed in claim 16. The spray plaster spray device according to the invention is specified in claim 22, the method for producing a polymer fiber nonwoven material is specified in claim 23, the polymer nonwoven material is specified in claim 24. Uses are specified in accordance with claims 26 and 27. Advantageous embodiments with expedient further embodiments of the invention are indicated in the respective dependent claims.

The plaster preparation comprises at least one resorbable polymer of at least 3, in particular, different, monomers, in particular a polymer comprising monomers selected from the group consisting of trimethylene carbonate, glycolide, lactide, in particular (D.L-lactide), p-dioxanone, ε-caprolactone and/or butyrolactone, at least one readily vaporizable organic solvent.

If the plaster preparation is a spray plaster preparation, it preferably comprises at least one propellant.

The plaster preparation according to the invention surprisingly allows a high inherent viscosity, wherein a high viscosity in the sense of the invention means a viscosity >0.75 dl/g, usually >1 dl/g. (According to the present application, the viscosity is to be determined as 1 g of polymer per 1 ml of chloroform at 25° C.). The polymer films and polymer nonwoven materials that can be produced by means of the plaster preparation or respectively spray plaster preparation according to the invention exhibit, at low thickness (film: 10-50 pm; nonwoven material: 80-200 pm), sufficient adhesion to skin and wounds for the intended use, good extensibility (film: 200-1500%; nonwoven material 150-200%), with varying strengths (film: 0.4-15 N/mm²; nonwoven material: 0.6-0.8 N/mm²) and good water vapor permeability (film: 15-80 g/m²/h; nonwoven material 80-120 g/m²/h), particularly adapted to the skin (human or animal, dry or wet). The plaster preparation according to the invention can therefore be applied externally in a very thin layer, particularly as a spray plaster preparation. The aforementioned adhesion is such that the polymer film or polymer nonwoven material cannot be removed from its external application site without causing damage. Rather, the polymer film or the polymer nonwoven material can only be removed manually in small shreds at a time. In contrast, during washing/showering and careful drying of the skin, the polymer film or polymer nonwoven material remains intact and adheres. The plaster preparation can also be applied as a pump spray or by means of a brush or the like to the application site (skin/wound) to be provided with a plaster.

Resorbable polymers in the sense of the invention are polymers prepared by polymerization of at least three different monomers, wherein both the polymers and the monomers used are substantially degraded by hydrolysis in vivo and in vitro. The polymers and their degradation products are medically safe and non-allergenic. The monomers are further metabolized in vivo via the citric acid cycle and fatty acid metabolism, respectively.

Suitable polymers contain in particular trimethylene carbonate, glycolide, lactide, in particular D,L-lactide, p-dioxanone, ε-caprolactone and/or butyrolactone.

In a preferred embodiment, the resorbable polymer contains trimethylene carbonate, in particular lactide, in particular D,L-lactide, ε-caprolactone and trimethylene carbonate, particularly preferably the terpolymer comprises lactide, in particular D,L-lactide, ε-caprolactone and trimethylene carbonate. This has the additional advantage that the polymers, with a glass transition point of 22° C.-37° C., are easily adapted to wound geometries and promote wound healing. The continuous release of the monomers as well as the degradation of the polymers to monomers promotes wound healing over a longer period of time. This effect can be enhanced by reapplication.

The polymer may preferably consist of 60 to 90% w/w D,L-lactide, 5 to 35% w/w ε-caprolactone and 5 to 35% w/w trimethylene carbonate, particularly 70 to 85% w/w D,L-lactide, 5 to 20% w/w ε-caprolactone and 5 to 20% w/w trimethylene carbonate. These compositions are particularly advantageous because amorphous structures are formed, which give rise to elastic and plastic behavior. Thus, mobility is maintained for wound areas covered with the plaster, e.g., on joints.

In a further embodiment, the polymer preferably has an inherent viscosity between 0.3 dl/g and 2.5 dl/g, in particular, between 0.75-1.6, very preferably between 0.9-1.6 dl/g (viscosity in the present application in each case measured with 1 mg of polymer per 1 ml of chloroform at 25° C.). This viscosity has the additional advantage that the resulting film is sufficiently mechanically stable and exhibits the desired degradation properties.

In a further embodiment, the polymer preferably has a monomer content of between 0.5 and 10% w/w, in particular a monomer content of between 3 and 8% w/w. This has the added advantage of promoting wound healing by releasing the monomers and giving the film the desired plastic, elastic behavior. The continuous release of monomers over a longer period of time promotes wound healing (over several days).

According to a further aspect of the invention, the plaster preparation can additionally comprise at least one further resorbable polymer, in particular a poly-ε-caprolactone, polylactide, polyglycolide, polyethylene glycol and/or at least one non-resorbable polymer, in particular from the group consisting of polyacrylates and polyurethanes, wherein the non-resorbable polymers are admixed in particular as a blend with a preferred proportion of 1-50% based on the terpolymer. This has the added advantage of increasing adhesion and improving flow properties (when creating a film).

Readily vaporizable organic solvents in the sense of the invention are organic solvents with an evaporation number (VD) according to DIN 53170 of <35, in particular, <10. Suitable organic solvents are particularly selected from the group consisting of acetone, methyl acetate, ethyl acetate, halogenated hydrocarbons, cyclopentane and/or mixtures thereof, particularly preferred are ethyl acetate, methyl acetate and/or acetone. Furthermore, organic solvents DMSO and N-pyrrolidone are suitable.

The resorbable polymer may be present dissolved in the organic solvent.

Propellants in the sense of the invention are gaseous or gas-emitting compounds by means of which other substances, in particular other liquids and/or gases, are transported and/or atomized. Suitable propellants include dim ethyl ether, methane, ethane, propane, butane, pentane, low-boiling (<50° C.) halogenated hydrocarbons, noble gases, air and/or CO2.

In another embodiment of the invention, the plaster preparation according to the invention may comprise a composition of 5-25% w/w resorbable polymer of at least 3 (different) monomer units and 75-95% w/w organic solvent.

In the case of the embodiment of the plaster preparation as a spray plaster preparation, in a further embodiment of the invention, said plaster preparation may comprise a composition of 2.8 to 7% w/w resorbable polymer of at least three (different) monomer units, 31.5 to 65% w/w organic solvent and 30 to 64% w/w propellant.

According to a further aspect of the invention, the plaster preparation may additionally have at least one disinfectant, in particular an antibacterial active substance, in particular polyhexanide, phenoxyethanol, iodine, peroxides, and/or silver. The plaster preparation may have disinfectants in a proportion by weight of 0.05% w/w-5% w/w, in particular 0.1% w/w-2% w/w.

Quite surprisingly, it has been found that the plaster preparation can be used to achieve a further increase in the extensibility of the polymer film that is producible or respectively produced therefrom by adding a disinfectant. For example, by adding a weight proportion of only 1% w/w of phenoxyethanol to the plaster preparation, a polymer film can be produced the extensibility of which is up to 1500%. The polymer film is particularly easy to deform repeatedly, and even after being stretched by, for example, 500%, it can automatically and elastically return to its original shape, either completely or almost completely. The modulus of the polymer film that is producible/produced from the plaster preparation is so small that the polymer membrane is imperceptible or barely perceptible to the wearer when applied externally to the skin. This makes it particularly comfortable to wear, particularly when applied close to the joint. In addition, undesired detachment of the polymer film from the application site can be counteracted even more efficiently in the event of repetitive stretching of the polymer film accompanied by movement of the person/animal supplied with the polymer film. At the same time, the durability of the plaster preparation can be improved and a beneficial antibacterial property of the polymer film produced therefrom can be realized.

Other additives can be cooling, analgesic, nourishing, or virucidal.

According to a further aspect of the invention, the plaster preparation may additionally contain biologically active additives, such as antibacterial substances, hemostatic substances, cytokines, growth factors, in particular, TGFß, anesthetics and/or biologically non-active additives, such as UVA and or UVB filters, dyes, other polymers, adhesives.

The invention also relates to a polymer film produced by means of spraying the plaster preparation according to the invention.

Polymer films produced by means of the plaster preparation according to the invention, in particular spray plaster preparation, exhibit, at low thickness (10-50 pm), sufficiently good adhesion to skin, fur and wounds for the intended use, good extensibility (200-1000%) and high strengths (0.4-15 N/mm²) and good water vapor permeability (15-80 g/m²/h), in particular, adapted to the skin. Polymer films according to the invention therefore have a high level of comfort when applied to the skin and accelerated wound healing due to the release of lactate.

Preferably, in one embodiment, the polymer film has a monomer content of <10% w/w. This has the additional advantage of achieving the mechanical characteristics desired for the intended use as a plaster and does not exhibit brittleness in conjunction with plastic and elastic behavior.

In one embodiment, the polymer film preferably has an E-modulus between 1.5 and 1000 N/mm², in particular, between 1.7 and 450 N/mm². This has the added advantage that the film is not stiff. This promotes sufficient adhesion to the skin.

In one embodiment, the polymer film has an extensibility between 100 to 1500%, particularly between 250 to 1000%. This has the additional advantage that the material is sufficiently flexible even for areas of the body that are difficult to cover geometrically, particularly in the area of joints.

The polymer film preferably has a strength of between 0.1 and 30 N/mm², in particular, between 1 and 20 N/mm². This has the additional advantage that the material does not tear even when applied close to the joint and during movements.

According to another advantageous embodiment, the polymer film has a thickness between 5 and 30 pm (measured after drying). Polymer films of this thickness are particularly comfortable to wear and also allow for a better air and moisture exchange. This promotes wound healing.

The invention also relates to a spray plaster spray device for spraying the spray plaster preparation according to the invention, comprising a housing with a spray head for initiating the spraying process, in particular a cylindrical housing, with a longitudinal axis. The housing comprises a reservoir for the spray plaster preparation and a propellant channel for a propellant of the spray plaster preparation with a diameter dₜ. The propellant channel extends in a first area in the direction of the longitudinal axis in the housing as a riser tube and extends in a second area in the spray head at an angle radially between the geodesic face side of the riser tube and a spray opening of the spray head.

The second area has a diameter d₂, which is smaller than the diameter d₁ of the first area. The second area (d2) opens into a mixing area with a diameter d₃. The mixing area is radially adjacent to the second area and connects the second area to a spray opening. A channel of the reservoir with a diameter extends in the direction of the longitudinal axis and opens into the mixing area and wherein its diameter d₄ is larger than the diameter d₃. The spray opening has a relief area increasing in an expanding cone-shaped manner with a maximum diameter d₅, wherein the relief area extends between the mixing area and the spray opening.

The spray plaster spray device according to the invention is accompanied by the advantage that the spray plaster preparation according to the invention is surprisingly also suitable, by spraying by means of the spray plaster spray device, for producing polymer fibers which can be deposited as a polymer fiber nonwoven material on a surface, in particular a skin (human or animal, dry or wet) or wound. This is accompanied by the advantage that the plaster preparation according to the invention can be applied as a fiber-containing sheet structure to a wound, for example a chronic wound, to reduce wound pain, reduce risk of maceration and/or infection in a wound, particularly in a chronic wound. Further, the polymer fiber nonwoven material according to the invention may be advantageous for stimulating vascularization in a wound, particularly in a chronic wound, and/or for use in building up the epidermis and dermis in a wound.

The invention also relates to a method for producing a polymer fiber nonwoven material comprising polymer fibers, wherein the spray plaster preparation according to the invention is sprayed by means of the spray plaster spray device according to the invention, wherein a propellant of the spray plaster preparation is forced into a mixing area of a propellant channel at a high speed, and wherein a polymer made of at least 3 monomers of the spray plaster preparation and a solvent of the spray plaster preparation is transported into the mixing area by means of a channel (from a reservoir) and is mixed with the propellant flowing at high speed and wherein the mixture obtained is transported into a relief space of a spray opening, wherein the solvent is vaporized as it exits the spray opening, wherein polymer fibers are formed, and wherein a polymer fiber nonwoven material having the polymer fibers is deposited on a surface, in particular on a human or animal skin surface, in particular on a wound.

The thickness of the polymer fiber nonwoven material to be produced can be selected by choosing the spraying time and repeating the spraying process as required. The fiber strength of the nonwoven material-forming polymer fibers can be influenced by the pressure in the spray plaster spray device, the polymer concentration, and the maximum opening method of the spray opening (=maximum inner diameter). The greater the pressure of the propellant and the smaller the maximum opening width, the smaller the fiber thickness and vice versa.

The invention further relates to a polymer fiber nonwoven material produced according to the method of the invention. The polymer fiber nonwoven material according to the invention is accompanied by the advantage that, surprisingly, the fibers can adapt optimally to the wound bed. They are also virtually solvent-free, which allows a less painful treatment when spraying the fibers. The nonwoven material structure also allows wound fluid to be better absorbed and wound secretions to be transported to the outside, which is particularly advantageous in the case of highly exuding wounds. In addition, the ventilation of the application site supplied with the polymer fiber nonwoven material can be improved.

According to an embodiment, the polymer fiber nonwoven material according to the invention preferably has a water vapor permeability of between 80 and 200 ml/m²/h. The polymer fiber nonwoven material according to the invention is accompanied by the advantage that fluid from weeping wounds can be drained more easily.

The invention also relates to the use of the plaster preparation according to the invention for topical treating of human or animal skin, in particular wound treatment of epidermal and dermal wounds, in particular abrasions, cuts, burn wounds and chronic wounds, in particular ulcer wounds, in particular pressure-induced ulcer (decubital ulcer), arterial ulcer, venous ulcer, mixed arterial-venous ulcer, ulcus cruris, arterial ulcus cruris, venous ulcus cruris, ulcer in diabetic foot syndrome and post-traumatic ulcer.

The invention also relates to the use of the plaster preparation according to the invention for use in redness of the skin, particularly in sunburn, for sun protection, in cosmetic applications, particularly for camouflage, or as a resorbable adhesive. Suitable dyes can be added to the plaster preparation for camouflage.

When used as a sunscreen, known per se and common UVA and UVB filters are added to the plaster preparation.

Further advantages of the invention can be found in the description and the drawings. The embodiments shown and described are not to be understood as an exhaustive list, but, rather, have an exemplary character for the description of the invention.

In each of the following examples, a standard spray head (=“standard nozzle”) with the designation “Kosmos” and with valve PV22210-200149 from Precision Dispensing Solutions Europe GmbH was used. The diameter specified in each case refers to the maximum opening width (=d5 in FIG. 1 of the drawing) of the standard nozzle.

Figures in round brackets are mass fractions in % w/w. It should be noted that viscosity values in the present application were each measured with 1 mg of polymer per 1 ml of chloroform at 25° C.

### Example 1

A spray plaster preparation containing poly-D,L-lactide-trimethylene carbonate-ε-caprolactone (75/15/10): Polymer at 7% w/w; Ethyl acetate at 63% w/w; Dimethyl ether at 30% w/w. The spray plaster preparation was placed in a pressure can (standard nozzle with diameter: 0.91 mm) in accordance with FIG. 1 and sprayed at a distance of approximately 10 cm and a pressure of approximately 5.1 bar (at 20° C.). A transparent polymer film with a thickness of about 12 pm and a water vapor permeability of about 30 ml/m²/h was formed. If the spray time is increased, the film thickness increases. At a 40 pm film thickness, the water vapor permeability was approximately 18 ml/m²/h.

When sprayed onto a moist piece of meat tempered to 37° C., a polymer film was formed within approximately 1-2 min that adhered sufficiently firmly (could not be displaced and remained in place during movement) for use as a wound plaster.

When applied to tempered, moist meat, the polymer film was removed with tweezers after approximately 1 to 2 minutes.

Even on human skin (both dry and wet), a layer was formed that could not be displaced and remained adherent during movement.

By means of uniaxial tensile tests, the following mechanical values were determined: Modulus of elasticity (E-modulus) in the linear range (DIN 53457), the tensile strength as tensile stress at break (N/mm²) and the elongations (%) at which the individual stresses occurred. The measurements were made at 20° C. E-modulus is approximately 250 N/mm². Elongation is approximately 350%. Tensile strength is approximately 7 N/mm².

The polymer film exhibits sufficient strength at low thickness for use as a plaster, a good adhesion to skin or wounds, and has a high-water vapor permeability, particularly comparable to the water vapor permeability of skin, without the need for further additives.

### Example 2

A spray plaster preparation containing poly-D,L-lactide-trimethylene carbonate-ε-caprolactone (75/15/10): Polymer at 10% w/w [8% w/w]; Ethyl acetate: 90% w/w [92% w/w]. The spray plaster preparation was placed in a pressure can with standard nozzle (with diameter: 0.91 mm) in accordance with FIG. 1 and sprayed with compressed air at a distance of approximately 10 cm and a pressure of approximately 5 bar. A transparent polymer film with a thickness of about 12 pm [10 pm] and a water vapor permeability of about 25 ml/m²/h [40 ml/m²/h] is formed. Uniaxial tensile tests yielded the following mechanical values: E-modulus at approximately 700 N/mm²; Elongation at approximately 300%; Tensile strength at approximately 11 N/mm².

The polymer film exhibits sufficient strength at low thickness for use as a plaster, good adhesion to skin or wounds, and has a high-water vapor permeability, particularly comparable to the water vapor permeability of skin, without the need for further additives.

### Example 3

A spray plaster preparation containing poly-D,L-lactide-trimethylene carbonate-ε-caprolactone (75/15/10) and violet dye “D&C Violet No. 2” to make the sprayed surface visible: Polymer at 3.6% w/w; Violet dye at 0.0045% w/w; Ethyl acetate at 41.3955% w/w; Dimethyl ether at 55% w/w. The spray plaster preparation was placed in a pressure can with standard nozzle (with diameter: 0.91 mm) in accordance with FIG. 1 and sprayed at a distance of 10 cm and a pressure of approximately 5.1 bar (at 20° C.). A violet, but still transparent polymer film with a thickness of about 15 μm and a water vapor permeability of about 41 ml/m²/h is formed. Uniaxial tensile tests yielded the following mechanical values: E-modulus at approximately 616 N/mm²; Elongation at approximately 348%; Tensile strength at approximately 8.7 N/mm².

The polymer film exhibits sufficient strength at low thickness for use as a plaster, a good adhesion to skin or wounds, and has a high-water vapor permeability, particularly comparable to the water vapor permeability of skin, without the need for further additives.

### Example 4

A spray plaster preparation containing poly-D,L-lactide-trimethylene carbonate-ε-caprolactone (75/15/10): Polymer at 3.5% w/w; Ethyl acetate at 31.5% w/w; Dimethyl ether at 65% w/w. The spray plaster preparation was placed in a pressure can with standard nozzle (with diameter: 0.91 mm) in accordance with FIG. 1 and sprayed at a distance of approximately 10 cm and a pressure of approximately 5.1 bar (at 20° C.). A transparent polymer film with a thickness of approximately 17 pm and a water vapor permeability of approximately 62 ml/m²/h is formed. Even on human skin (both dry and wet), a very firmly adherent layer is formed. Due to the high propellant gas content, many small bubbles form during spraying, but a polymer film still forms. Uniaxial tensile tests yielded the following mechanical values: E-modulus at approximately 416 N/mm²; Elongation at approximately 355%; Tensile strength at approximately 7.5 N/mm².

The polymer film exhibits a high strength with low thickness, a good adhesion to skin or wounds, and has a high-water vapor permeability, particularly comparable to the water vapor permeability of skin, without the need for further additives.

### Example 5

A spray plaster preparation containing poly-D,L-lactide-trimethylene carbonate-ε-caprolactone (75/15/10) with cyanoacrylate: Polymer at 3.6% w/w; Cyanoacrylate at 0.045% w/w; Ethyl acetate at 41.355% w/w; Dimethyl ether at 55% w/w. The spray plaster preparation was placed in a pressure can with standard nozzle (with diameter: 0.91 mm) in accordance with FIG. 1 and sprayed at a distance of approximately 10 cm and a pressure of approximately 5.1 bar (at 20° C.). A transparent polymer film with a thickness of approximately 14 pm and a water vapor permeability of approximately 29 ml/m²/h is formed. Even on human skin (both dry and wet), a very firmly adherent layer is formed. Uniaxial tensile tests yielded the following mechanical values: E-modulus: approximately 248 N/mm²; Elongation at approximately 486%; Tensile strength at approximately 14.3 N/mm².

The polymer film exhibits a high strength with low thickness, a good adhesion to skin or wounds and has a high-water vapor permeability, particularly comparable to the water vapor permeability of skin, without the need for further additives.

### Example 6

A spray plaster preparation containing poly-D,L-lactide-trimethylene carbonate-ε-caprolactone (75/15/10): Polymer at 13% w/w in ethyl acetate at 87% w/w; Polymer at 13% w/w in ethyl acetate/acetone mixture 1:1 at 87% w/w; Polymer at 16% w/w in acetone at 86% w/w. The spray plaster preparation was placed in a pressurized can with a spray head according to the invention in accordance with FIG. 1 and sprayed at a distance of approximately 30 cm and an air pressure of approximately 5 bar. Whitish polymer fibers with an average thickness of approximately (A) 850 nm (B) 1 pm (C) 2 pm were formed. The polymer fibers formed a polymer fiber nonwoven material according to the invention which was also obtained with a standard nozzle (with diameter: 0.91 mm) at low pressure (about 1 bar). The polymer fiber nonwoven material adheres well to human skin. A very firmly adherent polymer fiber nonwoven material also formed on human skin (both dry and wet).

The polymer nonwoven material (A) had a water vapor permeability of approximately 111 ml/m²/h. Uniaxial tensile tests yielded the following mechanical values: E-modulus at approximately 21 N/mm²; Elongation at approximately 184%; Tensile strength at approximately 0.82 N/mm².

The polymer nonwoven material exhibits a high strength with a low thickness of 120 pm, a good adhesion to skin or wounds, and has a high-water vapor permeability, particularly comparable to the water vapor permeability of skin, without the need for further additives.

### Example 7

A spray plaster preparation containing poly-D,L-lactide-trimethylene carbonate-ε-caprolactone (75/15/10): Polymer at 15% w/w in ethyl acetate at 85% w/w; Polymer at 17% w/w in ethyl acetate at 83% w/w; Polymer at 19% w/w in ethyl acetate at 81% w/w. The spray plaster preparation was placed in a pressurized can with a spray head according to the invention in accordance with FIG. 1 and sprayed at a distance of approximately 30 cm and an atmospheric pressure of approximately 8 bar. Whitish fibers with an average thickness of approximately (A) 900 nm (B) 1 pm (C) 2.5 pm were formed. A polymer fiber nonwoven material according to the invention was formed. The polymer fiber nonwoven material adhered to human skin in a manner suitable for use as a plaster or skin protector.

### Example 8

A spray plaster preparation containing poly-D,L-lactide-trimethylene carbonate-ε-caprolactone (75/15/10): Polymer at 3.6% w/w; Phenoxyethanol at 0.45% w/w; Ethyl acetate at 40.95% w/w; Dimethyl ether at 55% w/w. The spray plaster preparation was placed in a pressurized can with a standard nozzle (0.91 mm) in accordance with FIG. 1 and sprayed at a distance of approximately 10 cm and a pressure of approximately 5.1 bar (at 20° C.). A transparent polymer film with a thickness of about 15 μm and a water vapor permeability of about 37 ml/m²/h was formed. Even on human skin (both dry and wet), a very firmly adherent layer is formed. Due to the high propellant gas content, many small bubbles form during spraying, but a polymer film still forms. Uniaxial tensile tests yielded the following mechanical values: E-modulus at approximately 4 N/mm²; Elongation at approximately 770%; Tensile strength at approximately 2.6 N/mm².

The polymer film surprisingly shows a very high extensibility at low thickness, a good adhesion to skin or wounds, and has a high-water vapor permeability, particularly comparable to the water vapor permeability of skin, without the need for further additives.

### BRIEF DESCRIPTION OF THE DRAWINGS

In the drawings:

FIG. 1 shows schematically the structure of a plaster spraying device in accordance with an embodiment;

FIG. 2 shows images of polymer fibers of polymer fiber nonwoven material produced according to Example 5, wherein the polymer fibers were produced from plaster preparations (A) and (C) by spraying by means of a plaster spraying device according to the invention;

FIG. 3 shows scanning electron micrographs of polymer fibers of a polymer fiber nonwoven material produced from plaster preparations (A), (B) and (C) according to Example 5 by means of the plaster spray device shown in FIG. 1; and

FIG. 4 shows scanning electron micrographs of polymer fiber nonwoven materials according to example 6 which were produced by means of a spray fiber spray device according to the invention.

### DETAILED DESCRIPTION OF THE PREFERRED EMBODIMENTS

In the figures, identical or similar components are each numbered with the same reference numerals. The embodiments shown in the figures are merely exemplary for the description of the invention and are not to be understood as limiting.

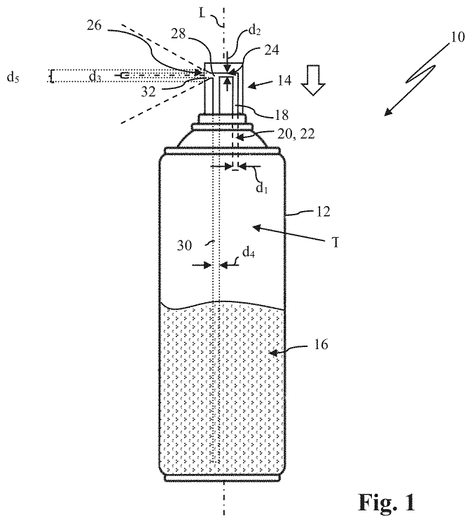

FIG. 1 schematically shows the structure of a pressurized spray plaster spray device 10 for spraying a plaster preparation according to the invention, in particular, for producing of polymer fiber nonwoven material of the plaster preparation according to a method for producing the polymer fiber nonwoven material according to the invention.

The spray plaster spray device 10 comprises a housing 12 with a movably arranged spray head 14 for initiating the spraying process. In particular, the housing 12 may be cylindrical or may have another shape. The housing 12 has a longitudinal axis L and comprises a reservoir 16 for the plaster preparation, a propellant channel 18 for a propellant T of the plaster preparation. The propellant 18 extends in a first area 20 in the direction of the longitudinal axis L in the housing as a riser tube 22 and extends in a second area 24 in the spray head 14 angled in a radial direction between the geodesic face side of the riser tube 22 and a spray opening 26 of the spray head 14. The first area 20 has an inner diameter d₁. The second area 24 has an inner diameter d₂, which is smaller than the diameter d₁ of the first area 20.

The second area 24 opens into a mixing area 28 having a diameter d₃, which is preferably less than or equal to the diameter d₂. The mixing area 28 connects in a radial direction to the second area 24 and fluidically connects the second area 24 to the spray opening 26.

A channel 30 of the reservoir 16 with a diameter d₄ extends in the axial direction and opens into the mixing area 28. Here, the diameter d₄ is larger than the diameter d₃ and the diameter d₂. Actuating the spray head creates a fluidic connection between the propellant T stored in the housing 12 and the spray opening 26. The remaining plaster preparation disposed in the housing 12 is drawn into the mixing area 28 by a subatmospheric suction mediated by the outflowing propellant.

The spray opening 26 has a relief area 32 expanding in an expansion cone-shaped manner, which extends in a direction radial to the longitudinal axis L between the mixing area 28 and the spray opening 26. The spray opening has a maximum inner diameter d₅ (=opening width).

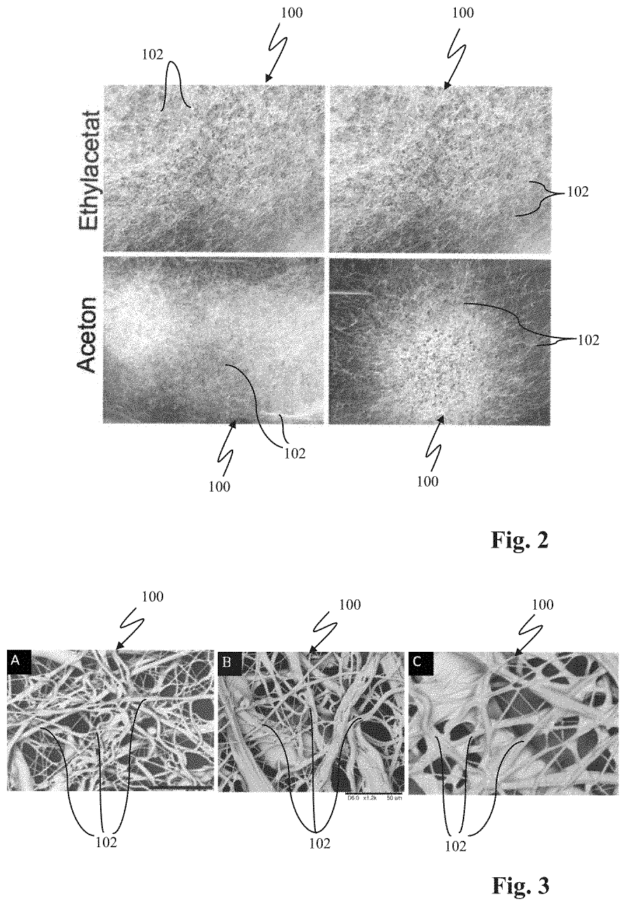

FIG. 2 shows photographic images of a polymer fiber nonwoven material 100 comprising a plurality of polymer fibers 102 produced according to Example 5 discussed above.

The polymer fibers 102 were produced by spraying plaster preparations: A, shown in the top row of images, and C, shown in the bottom row of images, by means of a spray fiber spray device 10 according to the invention in accordance with FIG. 1 onto a glass plate (not shown in FIG. 2) with a spraying distance of 30 cm. The polymer fibers 102 of the polymer fiber nonwoven materials 100 are clearly visible.

FIG. 3 shows scanning electron micrographs of polymer fiber nonwoven materials 100 each comprising polymer fibers 102 produced according to Example 5. The polymer fibers 102 were produced by spraying the plaster preparations A, B and C by means of the spray fiber spray device 10 in accordance with FIG. 1.

The left figure shows polymer fibers 102 produced from plaster preparation A. The middle figure shows polymer fibers 102 of plaster preparation B and the right figure shows polymer fibers 102 of plaster preparation C. All images are at a scale of 50 pm, i.e., a 1 cm section in the image corresponds to 50 pm in real life. The polymer fibers 102 exhibit a form fit typical of nonwoven materials as sheet materials by entanglement, cohesion, and/or adhesion.

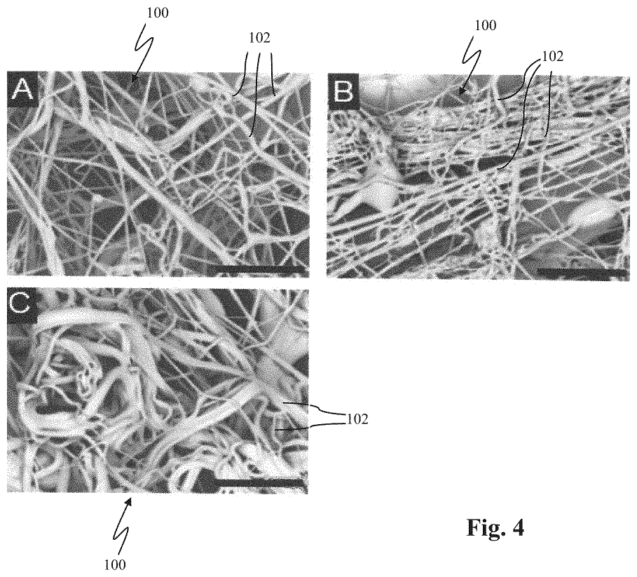

FIG. 4 shows scanning electron micrographs of polymer fiber nonwoven materials 100 according to example 6 described above, which were produced by means of a spray fiber spray device 10 according to the invention in accordance with FIG. 1. The scale is 50 μm. The polymer solutions were each sprayed at an internal pressure of the spray fiber spray device 10 of approximately 8 bar. The polymer concentrations were varied. Image A shows a polymer fiber nonwoven material 100 of solution A, image B shows a polymer fiber nonwoven material 100 of solution B, image C shows a polymer fiber nonwoven material 100 of solution C. The polymer fibers 102 or respectively polymer fiber nonwoven materials 100 exhibit adhesion to human skin sufficient even for wound closure.

What is claimed is:

1. A plaster preparation, comprising:
a) at least one resorbable polymer made of at least 3 monomer units, being a terpolymer, comprising monomers selected from the group consisting of:
trimethylene carbonate;
glycolide;
lactide;
p-dioxanone;
ε-caprolactone and/or butyrolactone; and
b) at least one readily vaporizable organic solvent.

2. The plaster preparation according to claim 1, wherein the lactide is D,L-lactide.

3. The plaster preparation according to claim 1, wherein the plaster preparation is a spray plaster preparation with at least one propellant.

4. The plaster preparation according to claim 1, wherein the resorbable polymer consists of: lactide being D,L-lactide, ε-caprolactone and trimethylene carbonate.

5. The plaster preparation according to claim 1, wherein the resorbable polymer consists of 60 to 90% w/w D,L-lactide, 5 to 35% w/w ε-caprolactone and 5 to 35% w/w trimethylene carbonate.

6. The plaster preparation according to claim 1, wherein the resorbable polymer consists of 70 to 85% w/w D,L-lactide, 5 to 20% w/w ε-caprolactone and 5 to 20% w/w trimethylene carbonate.

7. The plaster preparation according to claim 5, wherein the resorbable polymer has an inherent viscosity between 0.5 and 2.5 dl/g.

8. The plaster preparation according to claim 5, wherein the resorbable polymer has an inherent viscosity between 0.5 and 1.6 dl/g.

9. The plaster preparation according to claim 5, wherein the resorbable polymer has an inherent viscosity between 0.9 and 1.6 dl/g.

10. The plaster preparation according to claim 1, wherein the terpolymer has a monomer content between 0.5 and 10% w/w.

11. The plaster preparation according to claim 1, wherein the terpolymer has a monomer content between 3 and 8% w/w.

12. The plaster preparation according to claim 1, additionally comprising: at least one further resorbable polymer from the group consisting of poly-s-caprolactone, polylactide, polyglycolide, polyethylene glycol and poloxamer; and/or at least one non-resorbable polymer from the group consisting of polyacrylates, polycyanoacrylates, and polyurethanes; wherein the non-resorbable polymers are admixed as a blend.

13. The plaster preparation according to claim 1, additionally comprising: at least one further resorbable polymer from the group consisting of poly-E-caprolactone, polylactide, polyglycolide and polyethylene glycol; and/or at least one monomer, being cyanoacrylate, which polymerizes out on the skin to a non-resorbable polymer and yields a polymer blend of a resorbable polymer and a non-resorbable polymer.

14. The plaster preparation according to claim 1, wherein the organic solvent is selected from the group consisting of acetone, methyl acetate, ethyl acetate, halogenated hydrocarbons, DMSO, N-pyrrolidone, cyclopentane and/or mixtures thereof with cyclopentane.

15. The plaster preparation according to claim 1, wherein the terpolymer is present dissolved in the organic solvent.

16. The plaster preparation according to claim 1, wherein the propellant is dimethyl ether, methane, propane, butane, pentane, butene, low-boiling halogenated hydrocarbons, noble gases, air and/or CO2.

17. The plaster preparation according to claim 1, wherein the plaster preparation contains 5 to 30% w/w terpolymer and 70-95% w/w organic solvent.

18. The plaster preparation according to claim 1, wherein the plaster preparation contains 2.8 to 7% w/w terpolymer, 31.5 to 64.4% w/w organic solvent and 30 to 65% w/w propellant.

19. The plaster preparation according to claim 1, wherein the plaster preparation additionally has at least one disinfectant, being an antibacterial active substance, selected from the group consisting of polyhexanide, phenoxyethanol, iodine, peroxides, and/or silver.

20. The plaster preparation according to claim 1, wherein the plaster preparation additionally contains UVA and/or UVB filters, cytokines, growth factors being TGF-ß, local anesthetics, hemostatic or antibacterial active substances and/or biologically inactive additives being dyes or cyanoacrylates.

21. A polymer film produced by means of applying or spraying of the plaster preparation according to claim 1.

22. The polymer film according to claim 21, wherein the polymer film has an E-modulus between 1.5 and 1000 N/mm².

23. The polymer film according to claim 21, wherein the polymer film has an E-modulus between 1.7 and 450 N/mm².

24. The polymer film according to claim 21, wherein the polymer film has a monomer content of <10% w/w.

25. The polymer film according to claim 21, wherein the polymer film has an extensibility between 100 to 1500%.

26. The polymer film according to claim 21, wherein the polymer film has an extensibility between 250 and 600%.

27. The polymer film according to claim 21, wherein the polymer film has a strength between 0.1 and 30 N/mm².

28. The polymer film according to claim 21, wherein the polymer film has a strength between 1 and 20 N/mm².

29. The polymer film according to claim 21, wherein the polymer film has a thickness between 5 and 30 pm.

30. A spray plaster spray device for spraying the plaster preparation according to claim 3, comprising a housing with a spray head for initiating the spraying process, being a cylindrical housing, having a longitudinal axis, the housing comprising:
a reservoir for the plaster preparation, a propellant channel for a propellant of the plaster preparation with a diameter d₁, wherein the propellant channel extends in a first area in axial direction in the housing as a riser tube and extends in a second area in the spray head angled radially between the geodesic face side of the riser tube and a spray opening of the spray head;
wherein the second area has a diameter d₂ which is smaller than the diameter d₁ of the first area;
wherein the second area opens into a mixing area with a diameter d₃ which is smaller than the diameter d₂;
wherein the mixing area is radially adjacent to the second area and connects the second area to the spray opening;
wherein a reservoir channel with a diameter d₄ extends in the direction of the longitudinal axis and opens into the mixing area and wherein the diameter d₄ is larger than the diameter d₃, and wherein the spray opening has a relief area expanding in an expansion cone-shaped manner, which extends in a radial direction between the mixing area and the spray opening.

31. A method for producing a polymer fiber nonwoven material comprising polymer fibers, wherein the spray plaster preparation is sprayed by means of the spray plaster spray device in accordance to claim 30, wherein the propellant of the spray plaster preparation is forced at high velocity into the mixing area of the propellant channel; and
wherein the terpolymer of the spray plaster preparation and the solvent of the spray plaster preparation are transported into the mixing area by means of the reservoir channel and mixed with the propellant flowing at high speed; and
wherein the mixture obtained is transported into a relief space of the spray opening, wherein the solvent is vaporized as it exits the spray opening, wherein polymer fibers are formed, and wherein a polymer fiber nonwoven material having the polymer fibers is deposited on a surface, the surface being a human or animal skin surface.

32. A polymer fiber nonwoven material produced by the method according to claim 31.

33. The polymer fiber nonwoven material according to claim 32, wherein the polymer fiber nonwoven material has a water vapor permeability between 80 and 200 g/m²/h.

34. A use of the plaster preparation according to claim 1 for topical treating of human or animal skin for wound treatment of epidermal and dermal wounds being abrasions, cuts, burn wounds, chronic wounds and ulcer wounds.

35. A use of the plaster preparation according to claim 1 for external application in the case of reddening of the skin, sunburn, sun protection, cosmetic applications, camouflage and/or as a resorbable adhesive.
